(19) United States
(12) Patent Application Publication
(10) Pub. No.: US 2003/0086962 A1
(43) Pub. Date: May 8, 2003

(54) Cleansing products

(76) Inventors: Kelly J. Westerfield, Cincinnati, OH (US); Paul Robert Tanner, Maineville, OH (US); George Endel Deckner, Cincinnati, OH (US); Kenneth Eugene Kyte III, Lebanon, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/903,277

(22) Filed: Jul. 11, 2001

(51) Int. Cl. A61K 9/70

(52) U.S. Cl. 424/443

(57) ABSTRACT

The present invention is directed to a disposable, single use personal care cleansing product having a water insoluble substrate and from about 2% to about 400% of a composition, by weight of the substrate, comprising, (i) a silicone selected from the group consisting of dimethicone, dimethiconol, and mixtures thereof; and (ii) a lathering surfactant, wherein (i) and (ii) are in a ratio of from about 1:20 to about 5:1, and wherein the composition is combined prior to being added onto or impregnated into the substrate, and wherein the product is capable of generating an Average Lathering Volume of greater than or equal to about 30 ml.

The invention also relates to a method of manufacturing such cleansing product as well as a method of manufacturing the skin or hair with the cleansing product.

### FIELD

The present invention relates to disposable, personal care cleansing products useful for cleansing the skin or hair. The invention also encompasses a method for cleansing the skin or hair using the products of the present invention and also to methods for manufacturing these products.

### BACKGROUND

Personal cleansing products have traditionally been marketed in a variety of forms such as bar soaps, creams, lotions, and gels. These cleansing formulations have attempted to satisfy a number of criteria to be acceptable to consumers. These criteria include cleansing effectiveness, skin feel, mildness to skin, hair, and ocular mucosae, and lather volume. Ideal personal cleansers should lather well, gently cleanse the skin or hair, cause little or no irritation, and not leave the skin or hair overly dry after frequent use.

It is also highly desirable to deliver cleansing benefits from a disposable, single use personal care cleansing product. Disposable products are convenient because they obviate the need to carry cumbersome bottles, bars, jars, tubes, and other forms of cleansing products. Disposable products are also a more sanitary alternative to the use of a washcloth, or other cleansing implements intended for multiple reuse, because such implements may develop bacterial growth, unpleasant odors, and other undesirable characteristics related to repeated use.

Such disposable cleansing products should be made to satisfy the same criteria acceptable to consumers as were the traditional types of personal cleansing products. The disposable cleansing products should for example also cleanse effectively, have good skin feel, be mild to the skin, hair, and ocular mucosae, and have appropriate lather volume. Silicones are known to provide good skin feel. However, silicones are also known to suppress the lather of surfactants and cleansing products. Thus, it is desirable to formulate improved compositions due to the benefits of providing better feeling products which are highly desirable to consumers.

While the prior art disposable cleansing products have addressed some of the problems of achieving properties of an ideal personal cleansing product, they have not addressed the problems to the extent of or in the manner of the present invention. Therefore, there is a need for an improved disposable cleansing product.

### SUMMARY

The present invention is directed to a disposable, single use personal care cleansing product having a water insoluble substrate and from about 2% to about 400% of a composition, by weight of the substrate, comprising, (i) a silicone selected from the group consisting of dimethicone, dimethiconol, and mixtures thereof; and (ii) a lathering surfactant, wherein (i) and (ii) are in a ratio of from about 1:20 to about 5:1, and wherein the composition is combined prior to being added onto or impregnated into the substrate, and wherein the product is capable of generating an Average Lathering Volume of greater than or equal to about 30 ml.

The invention also relates to a method of manufacturing such cleansing product as well as a method of manufacturing the skin or hair with the cleansing product.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

All percentages are by weight of total composition unless specifically stated otherwise.

All ratios are weight ratios unless specifically stated otherwise.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The personal care cleansing product of the present invention are highly efficacious for cleansing the skin or hair, while providing a mildness benefit. The product has sufficient lather generation and provides the consumer with a rich, creamy and generous lather. After cleansing, the product leaves the surface of the skin or hair soft, and leaves the skin feeling smooth.

Without being limited by theory, it is believed that the substrate significantly contributes to the generation of lather. It is believed that this increase in lathering is the result of the surface action of the substrate. As a result, milder and significantly lower amounts of required surfactant can be used, resulting in a decrease in the drying effect of the skin or hair by the surfactants.

Finally, the substrate also enhances cleaning. The substrate can have differing textures on each side, e.g. a rough side and a smooth side. The substrate acts as an efficient lathering and exfoliating implement. By physically coming in to contact with the skin or hair, the substrate significantly aids in cleansing and removal of dirt, makeup, dead skin, and other debris.

Without wanting to be limited by theory, even with the addition of silicones, which are traditionally known to suppress lather, the applicants have found that by adding the particular silicones of the present invention in a certain ratio with a lathering surfactant, the resultant composition retains good lather qualities in addition to the aesthetic benefit of having a composition that feels “good” to consumers. The product thus has a reduction and/or elimination of poor feelings such as a dry draggy feeling left on the cleansed surface.

By a “lathering surfactant” is meant a surfactant, which when combined with water and mechanically agitated generates a foam or lather. Preferably, these surfactants should be mild, which means that these surfactants provide sufficient cleansing or detersive benefits but do not overly dry the skin or hair, and yet meet the lathering criteria described above.

The terms “disposable” or “single use”, are used herein in their ordinary sense to mean a product that is disposed or discarded after one usage event.

The term “substantially dry” as used herein means that the product is substantially free of water and generally feels dry to the touch. In certain embodiments, the products of the present invention may be substantially dry. “Substantially free of water” generally means the product comprises from about 0% about 20% by weight of water, preferably less than about 15% by weight of water, and more preferably less than about 12% by weight of water, the forgoing measured in a dry environment, (e.g., at 20-25° C. and less than or equal to about 50% relative humidity). One of ordinary skill in the art would recognize that the water content of a product such as in the present invention can vary with the relative humidity of the environment. In such embodiments wherein the substrate is substantially dry, the substrate is water-activated. The term “water-activated,” as used herein, means that the product is presented to the consumer in dry form to be used after wetting with water. It is found that these products produce a lather or are “activated” upon contact with water and further agitation.

The present invention is directed to a disposable, single use personal care cleansing product having a water insoluble substrate and from about from about 2% to about 400% of a composition, by weight of the substrate, comprising, (i) a silicone selected from the group consisting of dimethicone, dimethiconol, and mixtures thereof; and (ii) a lathering surfactant, wherein (i) and (ii) are in a ratio of from about 1:20 to about 5:1, and wherein the composition is combined prior to being added onto or impregnated into the substrate, and wherein the product is capable of generating an Average Lathering Volume of greater than or equal to about 30 ml.

Water Insoluble Substrate

The products of the present invention comprise a water insoluble substrate. By “water insoluble” is meant that the substrate does not dissolve in or readily break apart upon immersion in water. The water insoluble substrate is the implement or vehicle for delivering the lathering surfactant and the conditioning component of the present invention to the skin or hair to be cleansed and conditioned. Without being limited by theory, it is believed that the substrate, by providing mechanical agitation provides a lather generating effect. A wide variety of materials can be used as the substrate. The following nonlimiting characteristics are desirable: (i) sufficient wet strength for use, (ii) sufficient abrasivity, (iii) sufficient loft and porosity, (iv) sufficient thickness, and (v) appropriate size.

Nonlimiting examples of suitable insoluble substrates which meet the above criteria include nonwoven substrates, woven substrates, hydroentangled substrates, air entangled substrates, natural sponges, synthetic sponges, polymeric netted meshes, formed films, thermal bonded substrates, chemical bonded substrates, and the like. Preferred embodiments employ nonwoven substrates since they are economical and readily available in a variety of materials. By nonwoven is meant that the layer is comprised of fibers which are not woven into a fabric but rather are formed into a sheet, mat, or pad layer. The fibers can either be random (i.e., randomly aligned) or they can be carded (i.e. combed to be oriented in primarily one direction). Furthermore, the nonwoven substrate can be composed of a combination of layers of random and carded fibers.

Nonwoven substrates may be comprised of a variety of materials both natural and synthetic. By natural is meant that the materials are derived from plants, animals, insects or byproducts of plants, animals, and insects. By synthetic is meant that the materials are obtained primarily from various man-made materials or from natural materials which have been further altered. The conventional base starting material is usually a fibrous web comprising any of the common synthetic or natural textile-length fibers, or mixtures thereof.

Nonlimiting examples of natural materials useful in the present invention are silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, and mixtures thereof.

Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, polyurethane foam, and mixtures thereof. Examples of some of these synthetic materials include acrylics such as acrilan, creslan, and the acrylonitrile-based fiber, orion; cellulose ester fibers such as cellulose acetate, arnel, and acele; polyamides such as nylons (e.g., nylon 6, nylon 66, nylon 610, and the like); polyesters such as fortrel, kodel, and the polyethylene terephthalate fiber, dacron; polyolefins such as polypropylene, polyethylene; polyvinyl acetate fibers; polyurethane foams and mixtures thereof. These and other suitable fibers and the nonwoven materials prepared therefrom are generally described in Riedel, “Nonwoven Bonding Methods and Materials,” Nonwoven World (1987); The Encyclopedia Americana, vol. 11, pp. 147-153, and vol. 26, pp. 566-581 (1984); U.S. Pat. No. 4,891,227, to Thaman et al., issued Jan. 2, 1990; and U.S. Pat. No. 4,891,228 which are all incorporated by reference herein in their entirety.

See C.A. Hampel et al., The Encyclopedia of Chemistry, third edition, 1973, pp. 793-795 (1973); The Encyclopedia Americana, vol. 21, pp. 376-383 (1984); and G. A. Smook, Handbook of Pulp and Paper Technologies, Technical Association for the Pulp and Paper Industry (1986); which are incorporated by reference herein in their entirety, for information regarding nonwoven substrates.

Substrates made from natural materials useful in the present invention can be obtained from a wide variety of commercial sources. Nonlimiting examples of suitable commercially available paper layers useful herein include Airtex^{R}, an embossed airlaid cellulosic layer having a base weight of about 71 gsy, available from James River, Green Bay, Wis.; and Walkisoft^{R}, an embossed airlaid cellulosic having a base weight of about 75 gsy, available from Buckeye Technologies U.S.A., Mount Holly, N.C.

Methods of making nonwoven substrates are well known in the art. Generally, these nonwoven substrates can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The resulting layer, regardless of its method of production or composition, is then subjected to at least one of several types of bonding operations to anchor the individual fibers together to form a self-sustaining web. In the present invention the nonwoven layer can be prepared by a variety of processes including hydroentanglement, thermally bonding or thermo-bonding, and combinations of these processes. Moreover, the substrates of the present invention can consist of a single layer or two or more, or otherwise multiple layers. In addition, a multilayered substrate can include films and other nonfibrous materials.

Nonwoven substrates made from synthetic materials useful in the present invention can also be obtained from a wide variety of commercial sources. Nonlimiting examples include hydroentangled substrates in a Nubtex pattern, containing about 50% rayon and 50% polyester and having a basis weight of about 50 grams per square meter (gsm) and 70% rayon and 30% polyester and having a basis weight of about 54 grams per square meter (gsm), both provided by BBA of Simpsonville, S.C. See also preferred substrates described in U.S. Pat. No. 5,281,461, International Paper Company. Other suitable nonwoven layer materials useful herein include HEF 140-047, an apertured hydroentangled material containing about 50% rayon and 50% polyester, and having a basis weight of about 43 grams per square yard (gsy), available from Veratec, Inc., Walpole, Mass.; HEF 140-103, an apertured hydroentangled material containing about 50% rayon and 50% polyester, and having a basis weight of about 57 gsy, available from Veratec, Inc., Walpole, Mass.; NovonetR 149-616, a thermo-bonded grid patterned material containing about 100% polypropylene, and having a basis weight of about 50 gsy, available from Veratec, Inc., Walpole, Mass.; Novonet^{R} 149-801, a thermo-bonded grid patterned material containing about 69% rayon, about 25% polypropylene, and about 6% cotton, and having a basis weight of about 75 gsy, available from Veratec, Inc. Walpole, Mass.; Novonet^{R} 149-191, a thermo-bonded grid patterned material containing about 69% rayon, about 25% polypropylene, and about 6% cotton, and having a basis weight of about 100 gsy, available from Veratec, Inc. Walpole, Mass.; HEF Nubtex^{R} 149-801, a nubbed, apertured hydroentangled material, containing about 100% polyester, and having a basis weight of about 70 gsy, available from Veratec, Inc. Walpole, Mass.; Duralace^{R} 1236, an apertured, hydroentangled material, containing about 100% rayon, and having a basis weight from about 40 gsy to about 115 gsy, available from Chicopee, New Brunswick, N.J.; Duralace^{R} 5904, an apertured, hydroentangled material, containing about 100% polyester, and having a basis weight from about 40 gsy to about 115 gsy, available from Chicopee, New Brunswick, N.J.; Sontara 8868, a hydroentangled material, containing about 50% cellulose and about 50% polyester, and having a basis weight of about 60 gsy, available from Dupont Chemical Corp.

Alternatively, the water insoluble substrate can be a polymeric mesh sponge as described in European Patent No. EP 702550 Al published Mar. 27, 1996, incorporated by reference herein in its entirety. The polymeric sponge comprises a plurality of plies of an extruded tubular netting mesh prepared from a strong flexible polymer, such as addition polymers of olefin monomers and polyamides of polycarboxylic acids. Although these polymeric sponges are designed to be used in conjunction with a liquid cleanser, these types of sponges can be used as the water insoluble substrate in the present invention.

The substrate can be made into a wide variety of shapes and forms including flat pads, thick pads, thin sheets, ball-shaped implements, irregularly shaped implements, and having sizes ranging from a surface area of about a square inch to about hundreds of square inches. The exact size will depend upon the desired use and product characteristics. Especially convenient are square, circular, rectangular, or oval pads having a surface area of from about 1 in² to about 144 in², preferably from about 10 in² to about 120 in², and more preferably from about 30 in² to about 80 in², and a thickness of from about 1 mil to about 500 mil, preferably from about 5 mil to about 250 mil, and more preferably from about 10 mil to about 100 mil. A preferred embodiment has at least and up to four rounded corners. The substrate can be folded in half or in quarters to fit in desirable packages for dispensing.

The water insoluble substrates of the present invention can comprise two or more layers, each having different textures and abrasiveness. The differing textures can result from the use of different combinations of materials or from the use of different manufacturing processes or a combination thereof. A dual textured substrate can be made to provide the advantage of having a more abrasive side for exfoliation and a softer, absorbent side for gentle cleansing. In addition, separate layers of the substrate can be manufactured to have different colors, thereby helping the user to further distinguish the surfaces.

In certain embodiments, the substrates may contain apertures or openings in the cleansing surface of the substrate. Such apertures may be generally circular in shape or may be openings of other shapes, including squares, rectangles, trapezoids, diamonds, hexagons, irregular shapes and the like. Such apertures need not be uniform in size and shape, but preferably will be substantially uniform in both size and shape.

The apertures in the cleansing surface of the substrate may preferably range in average diameter between about 0.5 mm and 5 mm. More preferably, the apertures will range in size between about 1 mm to 4 mm in average diameter. Preferably no more than about 10% of the apertures in the cleansing surface of the substrate will fall outside these size ranges. More preferably no more than about 5% of the apertures in the cleansing surface will fall outside these size ranges. For apertures which are not circular in shape, the “diameter” of the aperture refers to the diameter of a circular opening having the same surface area as the opening of the non-circular shaped aperture.

For certain embodiments, within the cleansing surface of the substrate, the apertures may generally occur at a frequency of from about 0.5 to 12 apertures per straight linear centimeter. More preferably the apertures in the cleansing surface may occur at a frequency of from about 1.5 to 6 apertures per straight linear centimeter.

When used, such apertures are at least be placed within the cleansing surface of the substrate element herein. Such apertures need not protrude completely through to the surface of the substrate which is opposite to the cleansing surface. When two or more plies or layers are used to form the water-insoluble substrate, apertures may or may not be placed in all of the plies or layers. Frequently, as noted more fully hereinafter, the substrate may comprise two layers one of which includes the cleansing surface and is apertured. The other layer or ply which forms a backing for the substrate with the cleansing surface may not apertured.

Apertures may be formed in the cleansing surface of the water-insoluble substrate as such a substrate, or layer thereof, is being formed or fabricated. Alternatively, apertures may be formed in the cleansing surface after the substrate, or ply or layer thereof, comprising the cleansing surface has been completely formed.

Composition

The products of the present invention include from about 2% to about 400% of a composition, preferably from about 5% to about 200%, more preferably from about 10% to about 100%, by weight of the substrate. The composition further contains a silicone selected from the group consisting of dimethicone, dimethiconol, and mixtures thereof and a lathering surfactant. The ratio of the silicone to the lathering surfactant is from about 1:20 to about 5:1, preferably from about 1:10 to about 3:1, even more preferably from about 1:4 to about 1.5:1.

The composition is combined prior to being added onto or impregnated into the substrate.

Silicone

The products of the present invention include a composition containing dimethicones, dimethiconols, and their mixtures, all also known as polydimethylsiloxanes. Dimethicones correspond to the general chemical formula R₃SiO[R₂SiO]ₓSiR₃ wherein R is a methyl group and x is an integer chosen to achieve the desired molecular weight. Preferred dimethicones and dimethiconols are nonvolatile, and more preferred are nonvolatile dimethicones and dimethiconols with viscosities from 10 to 30,000 centistokes. Nonlimiting examples of commercially available dimethicones include the Vicasil® series sold by General Electric Company and the Dow Corning® 200 series sold by Dow Corning Corporation. Specific examples of dimethicones useful herein include Dow Corning® 200 fluids having viscosities of 50, 350, 1000, and 12,500 centistokes, respectively. Dimethiconols are hydroxy terminated dimethylsiloxanes. These materials can be represented by the general chemical formulas R₃SiO[R₂SiO]ₓSiR₂OH and HOR₂SiO[R₂SiO]ₓSiR₂OH wherein R is a methyl group and x is an integer chosen to achieve the desired molecular weight. Commercially available dimethiconols are typically sold as mixtures with dimethicone or cyclomethicone, examples of which include Dow Corning® 1401, 1402, 1403, and 1068 fluids sold by the Dow Corning Corporation.

Dimethicones, dimethiconols and mixtures thereof are also commercially available as water-continuous emulsions, examples of which include Dow Corning® 1664 and 1784 sold by the Dow Corning Corporation.

Lathering Surfactant

The composition of the present invention includes a silicone and a lathering surfactant. Preferably, the compositions contain from about 1.9% to about 380% by weight of the water insoluble substrate of a lathering surfactant. More preferably, the compositions contain from about 4.5% to about 180%, and even more preferably, from about 8% to about 80%, by weight of the water insoluble substrate of a lathering surfactant.

Lathering surfactants include anionic surfactants, nonionic surfactants, amphoteric surfactants, cationic surfactants, zwitterionic surfactants, and mixtures thereof.

Anionic Surfactant

Nonlimiting examples of anionic surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975 all of which are incorporated by reference herein in their entirety.

Nonlimiting examples of anionic surfactants include those selected from the group consisting of sarcosinates, sulfates, isethionates, taurates, phosphates, chelating surfactants, and mixtures thereof. Amongst the isethionates, the alkoyl isethionates are preferred, and amongst the sulfates, the alkyl and alkyl ether sulfates are preferred. The alkoyl isethionates typically have the formula RCO—OCH₂CH₂SO₃M wherein R is alkyl or alkenyl of from about 10 to about 30 carbon atoms, and M is a water-soluble cation such as ammonium, sodium, potassium and triethanolamine. Nonlimiting examples of these isethionates include those alkoyl isethionates selected from the group consisting of ammonium cocoyl isethionate, sodium cocoyl isethionate, sodium lauroyl isethionate, and mixtures thereof.

The alkyl and alkyl ether sulfates typically have the respective formulae ROSO₃M and RO(C₂H₄O)_{X}SO₃M, wherein R is alkyl or alkenyl of from about 10 to about 30 carbon atoms, x is from about 1 to about 10, and M is a water-soluble cation such as ammonium, sodium, potassium and triethanolamine. Another suitable class of anionic surfactants is the water-soluble salts of the organic, sulfuric acid reaction products of the general formula:

R₁—SO₃—M

wherein R₁ is chosen from the group consisting of a straight or branched chain, saturated aliphatic hydrocarbon radical having from about 8 to about 24, preferably about 10 to about 16, carbon atoms; and M is a cation. Still other anionic synthetic surfactants include the class designated as succinamates, olefin sulfonates having about 12 to about 24 carbon atoms, and b-alkyloxy alkane sulfonates. Examples of these materials are sodium lauryl sulfate and ammonium lauryl sulfate.

Other anionic materials useful herein are soaps (i.e. alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853, cited above.

Other anionic materials include phosphates such as monoalkyl, dialkyl, and trialkylphosphate salts.

Other anionic materials include alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and trialkanolamine (e.g., triethanolamine), preferred examples of which include sodium lauroyl sarcosinate, sodium cocoyl sarcosinate, and ammonium lauroyl sarcosinate.

Also useful are taurates which are based on taurine, which is also known as 2-aminoethanesulfonic acid. Examples of taurates include N-alkyltaurines such as the one prepared by reacting dodecylamine with sodium isethionate according to the teaching of U.S. Pat. No. 2,658,072 which is incorporated herein by reference in its entirety.

A type of anionic surfactant includes a chelating surfactant. The chelating surfactant is a salt and/or salts of a hydrophobically modified ethylenediaminetriacetic acid, including mixtures thereof, wherein the hydrophobically modified ethylenediamineacetic acids have a general structure as follows:

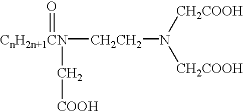

where n is from 1 to 40.

If unsaturation occurs, the hydrophobically modified group may be CₙH₂ₙ₋₁ where n is 2 to 40, and if further unsaturation occurs, the group may be CₙH₂ₙ₋₃ where n is 3 to 40 and so forth. The salt are the salt of one or more of the carboxylic acid groups. These compounds and methods of their preparation are described, for example, in U.S. Pat. No. 5,284,972, 5,177,243, U.S. Pat. No. 5,191,081, and 5,191,106, hereby incorporated by reference into the subject application. The synthesis, physical and physiological properties of the chelating surfactants, as well as examples of chelating surfactants, are also summarized in Inform, Vol. 6 no. 10, October 1995, by B. Parker et al.

The counterions which may be used for the chelating surfactant include but are not limited to sodium, potassium, ammonium, monoethanolamine, diethanolamine, triethanolamine, N-propylamine, isoproylamine, and tris(hydroxymethyl aminomethane).

Examples of chelating surfactants include sodium lauroyl ethylenediaminetriacetic acid, potassium cocoyl ethylenediaminetriacetic acid, triethanolamine myristoyl ethylenediaminetriacetic acid, sodium oleoyl ethylenediaminetriacetic acid, and mixtures thereof.

Nonlimiting examples of preferred anionic surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl sarcosinate, sodium lauroyl ethylenediaminetriactetic acid, potassium cocoyl ethylenediaminetriacetic acid, triethanolamine myristoyl ethylenediaminetriacetic acid, and mixtures thereof.

Nonionic Surfactants

Nonlimiting examples of nonionic surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonionic surfactants useful herein include those selected from the group consisting of alkyl glucosides, alkyl polyglucosides, polyhydroxy fatty acid amides, alkoxylated fatty acid esters, sucrose esters, amine oxides, and mixtures thereof.

Alkyl glucosides and alkyl polyglucosides are useful herein, and can be broadly defined as condensation products of long chain alcohols, e.g. C8-30 alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the structural formula:

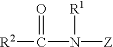

wherein: R¹ is H, C₁-C₄ alkyl, 2-hydroxyethyl, 2-hydroxy-propyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polhydroxyhydrocarbyl moiety having a linear hydrocarbyl chain with a least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Other examples of nonionic surfactants include amine oxides. Amine oxides correspond to the general formula R₁R₂R₃NO, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyl-dodecylamine oxide, oleyidi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Nonlimiting examples of preferred nonionic surfactants for use herein are those selected form the group consisting of C8-C14 glucose amides, C8-C14 alkyl polyglucosides, sucrose cocoate, sucrose laurate, lauramine oxide, cocoamine oxide, and mixtures thereof.

Cationic Surfactants

The products of the present invention can also optionally contain one or more cationic surfactants. Preferably, these materials are selected so as not to interfere with the overall lathering characteristics of the cleansing products.

Nonlimiting examples of cationic surfactants useful herein are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Preferred cationic surfactants useful herein include those selected from the group consisting of dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, and mixtures thereof.

Amphoteric Surfactants

The term “amphoteric surfactant,” as used herein, is also intended to encompass zwitterionic surfactants, which are well known to formulators skilled in the art as a subset of amphoteric surfactants.

A wide variety of amphoteric surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Examples of sultaines and hydroxysultaines include materials such as cocamidopropyl hydroxysultaine (available as Mirataine CBS from Rhone-Poulenc).

Preferred for use herein are amphoteric surfactants having the following structure:

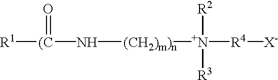

wherein R¹ is unsubstituted, saturated or unsaturated, straight or branched chain alkyl having from about 9 to about 22 carbon atoms. Preferred R¹ has from about 11 to about 18 carbon atoms; more preferably from about 12 to about 18 carbon atoms; more preferably still from about 14 to about 18 carbon atoms; m is an integer from 1 to about 3, more preferably from about 2 to about 3, and more preferably about 3; n is either 0 or 1, preferably 1; R² and R³ are independently selected from the group consisting of alkyl having from 1 to about 3 carbon atoms, unsubstituted or mono-substituted with hydroxy, preferred R² and R³ are CH₃; X is selected from the group consisting of CO₂, SO₃ and SO₄; R⁴ is selected from the group consisting of saturated or unsaturated, straight or branched chain alkyl, unsubstituted or monosubstituted with hydroxy, having from 1 to about 5 carbon atoms. When X is CO₂, R⁴ preferably has 1 or 3 carbon atoms, more preferably 1 carbon atom. When X is SO₃ or SO₄, R⁴ preferably has from about 2 to about 4 carbon atoms, more preferably 3 carbon atoms.

Examples of amphoteric surfactants of the present invention include the following compounds:

Cetyl dimethyl betaine (this material also has the CTFA designation cetyl betaine), cocamidopropylbetaine, and cocamidopropyl hydroxy sultaine.

Examples of other useful amphoteric surfactants are alkyliminoacetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-higher alkyl aspartic acids such as those produced according to the teaching of U.S. Pat. No. 2,438,091 which is incorporated herein by reference in its entirety; and the products sold under the trade name “Miranol” and described in U.S. Pat. No. 2,528,378, which is incorporated herein by reference in its entirety. Other examples of useful amphoterics include amphoteric phosphates, such as coamidopropyl PG-dimonium chloride phosphate (commercially available as Monaquat PTC, from Mona Corp.). Also useful are amphoacetates such as disodium lauroamphodiacetate, sodium lauroamphoacetate, and mixtures thereof.

The preferred anionic surfactant is selected from the group consisting of chelating surfactants, sarcosinates, sulfates, isethionates, phosphates, taurates, and mixtures thereof; wherein the preferred nonionic surfactant is selected from the group consisting essentially of amine oxides, alkyl glucosides, alkyl polyglucosides, polyhydroxy fatty acid amides, polyhydroxy fatty acid esters, sucrose esters, and mixtures thereof; and wherein the preferred amphoteric surfactant is selected from the group consisting essentially of betaines, sultaines, hydroxysultaines, alkyliminoacetates, iminodialkanoates, aminoalkanoates, and mixtures thereof.

Preferred surfactants for use herein also include the following, wherein the anionic surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl ethylenediaminetriactetic acid, potassium cocoyl ethylenediaminetriacetic acid, triethanolamine myristoyl ethylenediaminetriacetic acid, and mixtures thereof; wherein the nonionic surfactant is selected from the group consisting of lauramine oxide, cocoamine oxide, decyl polyglucose, lauryl polyglucose, sucrose cocoate, C12-14 glucosamides, sucrose laurate, and mixtures thereof; and wherein the amphoteric surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

The products of the present invention are capable of generating an Average Lathering Volume of greater than or equal to about 30 ml.

Lather Volume Test

The preferred products of the present invention may contain enough of a lathering surfactant to generate greater than or equal to about 30 ml, preferably greater than about 50 ml, more preferably greater than about 75 ml, and even more preferably greater than about 100 ml of Average Lather Volume. The Average Lather Volume is a measurement determined by the Lather Volume Test. This test provides a consistent volume measurement of the lather/foam generated by the articles described herein. The Lather Volume Test protocol is described as follows:

(1) Hands are washed with Ivory bar before conducting the test. This step removes any soils which may affect the accuracy of the measurement.

(2) The test article is held open in the non-dominant hand with the edges turned up.

(3) 10 ml. of water (medium hardness of about 8-10 grains per gallon) at 95° F. is added onto the test article via a 10 cc syringe or a Brinkmann repipetter.

(4) The lather is then generated by rubbing the test article with the dominant hand in a circular motion between the palms for 6 seconds (˜2 rotations per second), using moderate pressure (e.g., 4 oz.), and allowing the article to ball-up between the palms of the hand.

(5) The test article is then held open in the non-dominant hand and an additional 10 ml of water (medium hardness of about 8-10 grains per gallon) at 95° F. is added onto the test article via a 10 cc syringe or a Brinkmann repipetter. The wetted article is again rubbed with the dominant had (3 rotations) using moderate force (e.g, 4 oz.) so that the test article becomes balled-up between the palms.

(6) The test article is then opened and rubbed 5 times by holding one edge of the article in one hand and rotating the hand holding the other side to further activate lather.

(7) The test article is then flipped over and Step #6 is repeated using the other hand.

(8) The lather is gathered by holding the test article in a cupped hand and scraping the lather off the test article with the other hand, being careful to only scrape lather form the test article. The lather from the test article is placed into a graduated cylinder or beaker big enough to hold the generated lather. This procedure is repeated 5 times on the same test article, and the lather from each iteration is accumulated in the same graduated cylinder or beaker. The total accumulated lather from these iterations is designated as the Lather Volume.

(9) To achieve consistent results, the Average Lather Volume is reported as the average of three test sample replications of Steps 1-8.

Optional Ingredients

The products of the present invention may optionally contain a wide range of other optional ingredients which are added onto or impregnated into the substrate.

Conditioning Component

The products of the present invention may contain an oil soluble conditioning component or agent or a water soluble conditioning component or agent, or mixtures thereof, which are useful for providing a conditioning benefit to the skin or hair during the use of the product. Preferred levels include from about 1% to about 100%, preferably from about 2% to about 75%, and more preferably from about 3% to about 50%, by weight of said water insoluble substrate.

The oil soluble conditioning component is selected from one or more oil soluble conditioning components such that the weighted arithmetic mean solubility parameter of the oil soluble conditioning component is less than or equal to 10.5.

Nonlimiting examples of conditioning components useful as oil soluble conditioning components include those selected from the group consisting of mineral oil, petrolatum, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, silicones, vegetable oils, hydrogenated vegetable oils, polypropylene glycol C4-C20 alkyl ethers, di C8-C30 alkyl ethers, vitamin E and vitamin E derivatives, niacinamide derivatives, salicylic acid, and mixtures thereof.

The present invention can also optionally comprise water soluble conditioning components or agents. The water soluble conditioning component is selected from one or more water soluble conditioning components such that the weighted arithmetic mean solubility parameter of the water soluble conditioning component is greater than 10.5.

Nonlimiting examples of conditioning components useful as water soluble conditioning components include those selected from the group consisting of panthenol, niacinamide and its derivatives, polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof. Specific examples of useful water soluble conditioning components include materials such as urea; guanidine; glycolic acid and glycolate salts (e.g. ammonium and quaternary alkyl ammonium); lactic acid and lactate salts (e.g. ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, polypropylene glycols such as PPG-9, PPG-12, PPG-15, PPG-17, PPG-20, PPG-26, PPG-30, PPG-34; alkoxylated glucose; hyaluronic acid; and mixtures thereof. Also useful are materials such as aloe vera in any of its variety of forms (e.g., aloe vera gel), chitin, starch-grafted sodium polyacrylates such as Sanwet (RTM) IM-1000, IM-1500, and IM-2500 (available from Celanese Superabsorbent Materials, Portsmouth, Va.); lactamide monoethanolamine; acetamide monoethanolamine; and mixtures thereof. Also useful are propoxylated glycerols as described in propoxylated glycerols described in U.S. Pat. No. 4,976,953, to Orr et al., issued Dec. 11, 1990, which is incorporated by reference herein in its entirety.

In one embodiment preferred conditioning components are selected from the group consisting of esters of fatty acids, polyol polyesters, glycerin mono-esters, glycerin di-esters, glycerin trimesters, epidermal and sebaceous hydrocarbons, lanolin, mineral oil, vegetable oil, vegetable oil adduct, petrolatum, nonionic polymers, glycerin, glycerol, propylene glycol, polypropylene glycols, polyethylene glycols, ethyl hexanediol, hexylene glycols, other aliphatic alcohols, panthenol, urea cationic polymers, polyols, glycolic acid, lactic acid, silicone, vitamin E, vitamin E derivaties, salicylic acid, niacinamide, niacinamide derivatives, and mixtures thereof.

In embodiments wherein a conditioning component is used, the products preferably deposit greater than about 2.5 micrograms/cm² of the conditioning component to the skin or hair during use of the product.

Quantitation of the conditioning component deposited on the skin or hair can be measured using a variety of standard analytical techniques well known to the chemist of ordinary skill in the art. Such methods include for instance extraction of an area of the skin or hair with a suitable solvent followed by analysis by chromatography (i.e. gas chromatography, liquid chromatography, supercritical fluid chromatography, etc.), IR spectroscopy, UVNIS spectroscopy, mass spectrometry, etc. Direct measurements can also be made on the skin or hair by techniques such as IR spectroscopy, UVNIS spectroscopy, opacity measurements, fluoresce spectroscopy, ESCA spectroscopy, and the like.

In a typical method for measuring deposition, an embodiment of the present invention is wetted with water and squeezed and agitated to generate a lather. The product is then rubbed for approximately 15 seconds on a site, approximately about 25 cm² to about 300 cm², preferably about 50 cm² to about 100 cm², on the skin or head which has been demarcated using an appropriate indelible marker. The site is then rinsed for approximately 10 seconds and then allowed to air dry for approximately 10 minutes. The site is then either extracted and the extracts analyzed, or analyzed directly using any techniques such as those exemplified above.

Inorganic or Organic Salt

The products of the present invention may optionally contain from about 35% to about 200%, preferably from about 50% to about 150%, more preferably from about 50% to about 100%, by weight of the total surfactant active of an inorganic or organic salt having divalent metal counterions. Preferred materials include inorganic and organic salts of calcium, magnesium, zinc, and other divalent counterions. Examples of di-valent salts include, but are not limited to, calcium chloride, magnesium chloride, magnesium sulfate, magnesium stearate, calcium laurate, and mixtures thereof.

Active Ingredients

The products of the present invention may optionally contain a safe and effective amount of one or more active ingredients or pharmaceutically-acceptable salts thereof which can be added onto or impregnated into the substrate.

The term “safe and effective amount” as used herein, means an amount of an active ingredient high enough to modify the condition to be treated or to deliver the desired skin benefit, but low enough to avoid serious side effects, at a reasonable benefit to risk ratio within the scope of sound medical judgment. What is a safe and effective amount of the active ingredient will vary with the specific active, the ability of the active to penetrate through the skin, the age, health condition, and skin condition of the user, and other like factors.

The active ingredients useful herein can be categorized by their therapeutic benefit or their postulated mode of action. However, it is to be understood that the active ingredients useful herein can in some instances provide more than one therapeutic benefit or operate via more than one mode of action. Therefore, classifications herein are made for the sake of convenience and are not intended to limit the active ingredient to that particular application or applications listed. Also, pharmaceutically-acceptable salts of these active ingredients are useful herein. The following active ingredients are useful in the compositions of the present invention.

Anti-Acne Actives: Examples of useful anti-acne actives include the keratolytics such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate, and cholate.

Anti-Wrinkle and Anti-Skin Atrophy Actives: Examples of antiwrinkle and anti-skin atrophy actives include retinoic acid and its derivatives (e.g., cis and trans); retinol; retinyl esters; niacinamide and derivatives of niacinamide and nicotinic acid, salicylic acid and derivatives thereof; sulfur-containing D and L amino acids and their derivatives and salts, particularly the N-acetyl derivatives, a preferred example of which is N-acetyl-L-cysteine; thiols, e.g. ethane thiol; hydroxy acids, phytic acid, lipoic acid; lysophosphatidic acid, and skin peel agents (e.g., phenol and the like).

Non-Steroidal Anti-Inflammatory Actives (NSAIDS): Examples of NSAIDS include the following categories: propionic acid derivatives; acetic acid derivatives; fenamic acid derivatives; biphenylcarboxylic acid derivatives; and oxicams. All of these NSAIDS are fully described in U.S. Pat. No. 4,985,459 to Sunshine et al., issued Jan. 15, 1991, incorporated by reference herein in its entirety. Examples of useful NSAIDS include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Also useful are the steroidal anti-inflammatory drugs including hydrocortisone and the like.

Topical Anesthetics: Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Artificial Tanning Agents and Accelerators. Examples of artificial tanning agents and accelerators include dihydroxyacetaone, tyrosine, tyrosine esters such as ethyl tyrosinate, and phospho-DOPA.

Antimicrobial and Antifungal Actives: Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole.

Preferred examples of actives useful herein include those selected from the group consisting of salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, neocycin sulfate, and mixtures thereof.

Sunscreen Actives: Also useful herein are sunscreening actives. A wide variety of sunscreening agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of 2-ethylhexyl pmethoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991; these two references are incorporated by reference herein in their entirety. Exact amounts of sunscreens which can be employed will vary depending upon the sunscreen chosen and the desired Sun Protection Factor (SPF) to be achieved. SPF is a commonly used measure of photoprotection of a sunscreen against erythema. See Federal Register, Vol. 43, No. 166, pp. 38206-38269, Aug. 25, 1978, which is incorporated herein by reference in its entirety.

Nonlimiting examples of preferred actives useful herein include those selected from the group consisting of salicylic acid, benzoyl peroxide, niacinamide and niacinamide derivatives, cis-retinoic acid, trans-retinoic acid, retinol, retinyl palmitate, phytic acid, N-acetyl L-cysteine, azelaic acid, lipoic acid, resorcinol, lactic acid, glycolic acid, ibuprofen, naproxen, hydrocortisone, phenoxyethanol, phenoxypropanol, phenoxyisopropanol, 2,4,4,′-trichloro-2′-hydroxy diphenyl ether, 3,4,4′-trichlorocarbanilide, 2-ethylhexyl p-methoxycinnamic acid, oxybenzone, 2-phenylbenzimidozole-5-sulfonic acid, dihydroxyacetone, octocrylene, octyl salicylate, 4,4′-methoxy-t-butyldibenzoylmethane, zinc oxide, and mixtures thereof.

Other Optional Ingredients

The products of the present invention can contain a wide range of other optional components which are added onto or impregnated into the substrate. These additional components should be pharmaceutically acceptable. The CTFA Cosmetic Ingredient Handbook, Second Edition, 1992, which is incorporated by reference herein in its entirety, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference. Examples of these and other functional classes include: abrasives, absorbents, anticaking agents, antioxidants, vitamins, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, preservatives, propellants, reducing agents, skin bleaching agents, and sunscreening agents.

Also useful herein are aesthetic components such as fragrances, pigments, colorings, essential oils, skin sensates, astringents, skin soothing agents, and skin healing agents.

Cosmetic Powders

The compositions of the present invention can comprise one or more cosmetic powders. These cosmetic powders are useful for providing an improved sensory experience during use of the product and improving the feel of the skin or hair after use of the product. Cosmetic powders useful in the present invention include spherical or sphere-like, platelet, and irregularly shaped powders with average particle sizes ranging from 0.01 microns to 100 microns. Preferred cosmetic powders include spherical or sphere-like powders and platelet shaped powders with average particle sizes ranging from 0.1 to 50 microns.

Non-limiting examples of cosmetic powders useful in the present invention include powders made from boron nitride, cellulose triacetate, ethylene acrylic acid copolymer, mica, sericite, nylon-6, nylon-12, PMMA, polyethylene, PTFE, polypropylene, silicone resin, silk, talc, and TiO2. The cosmetic powders may also be coated with a surface coating to modify the behavior and sensory characteristics of the powder. Non-limiting examples of suitable coating materials include silicones, lecithin, amino acids, metal soaps, polyethylene, and collagen.

Preferred cosmetic powders useful in the present invention include spherical and sphere-like powders made from PTFE, aluminum starch octenylsuccinate, polyethylene, polypropylene, and polymethylsilsesquioxane silicone polymer, and platelet shaped powder made from L-lauroyl lysine.

Methods of Manufacture

The disposable, single use personal care cleansing products of the present invention may be manufactured by adding onto or impregnating into a water insoluble substrate a composition containing the particular ratio of silicone and lathering surfactant as in the present invention. It is important that the silicone and lathering surfactant be combined prior to being added onto or impregnated into the substrate. Examples of “combined prior” include the following: the silicone is added to the surfactant and mixed or milled together, the surfactant is added to the silicone and mixed or milled together, and the silicone and surfactant are simultaneously added to a mixing vessel and mixed or milled together. Additional examples of “combined prior” include the silicone is mixed or milled together with a portion of the surfactant, and then this premix is combined with the remainder of the surfactant and mixed or milled together.

Preferably, additional ingredients such as conditioning components and other ingredients are also added onto or impregnated into the substrate. In some embodiments, the product may subsequently be dried so that it is substantially dry. Alternatively, the product may be dried at one or more intermediate points during its manufacture. In other embodiments, some of the components which are added onto or impregnated into the substrate may be separately or simultaneously added onto or impregnated into the substrate. By “separately” is meant that the components can be added sequentially, in any order without first being combined together. By “simultaneously” is meant that the components can be added at the same time, with or without first being combined together.

For example, the lathering surfactants and silicone can be combined and then be added onto or impregnated into the water insoluble substrate followed by the conditioning agents, or vice versa. Alternatively, the combined lathering surfactants and silicone can be added onto or impregnated into the water insoluble substrate at the same time as the conditioning agents. Alternatively, the surfactants, silicone, and conditioning agents can be all combined together before adding onto or impregnating into the water insoluble substrate.

The silicone and lathering surfactant and any optional ingredients can be added onto or impregnated into the water insoluble substrate by any means known to those skilled in the art: for example, by spraying, laser printing, splashing, dipping, soaking, or coating.

For one embodiment wherein the substrate is substantially dry, when water or moisture is used or present in the manufacturing process, the resulting treated substrate is then dried so that it is substantially free of water. The treated substrate can be dried by any means known to those skilled in the art. Nonlimiting examples of known drying means include the use of convection ovens, radiant heat sources, microwave ovens, forced air ovens, and heated rollers or cans. Drying also includes air drying without the addition of heat energy, other than that present in the ambient environment. Also, a combination of various drying methods can be used.

Methods of Cleansing and Conditioning the Skin or Hair

The present invention also relates to a method of cleansing and conditioning the skin or hair with a personal cleansing product of the present invention.

For embodiments wherein the substrate is substantially dry, these methods comprise the steps of wetting with water a substantially dry, disposable, single use personal cleansing product comprising a water insoluble substrate, and a composition containing silicone and lathering surfactant, and contacting the skin or hair with said wetted product. Such products are wetted by immersion in water or by placing it under a stream of water. Lather is generated from the product by mechanically agitating and/or deforming the product either prior to or during contact of the product with the skin or hair. The resulting lather is useful for cleansing and conditioning the skin or hair. During the cleansing process and subsequent rinsing with water, the option ingredients such as conditioning agents and active ingredients are deposited onto the skin or hair. Deposition of conditioning agents and active ingredients are enhanced by the physical contact of the substrate with the skin or hair.

### EXAMPLES

The following examples further describe and demonstrate embodiments within the scope of the present invention. In the following examples, all ingredients are listed at an active level. The examples are given solely for the purpose of illustration and are not to be construed as limitations of the present invention, as many variations thereof are possible without departing from the spirit and scope of the invention. Ingredients are identified by chemical or CTFA name.

### Examples 1-6

The compositions of examples 1-6 are prepared by mixing the following ingredients together in the order shown at room temperature in a suitable vessel:

| | Weight Percent | | | | | |
|---|---|---|---|---|---|---|
| | Example | Example | Example | Example | Example | Example |
| Ingredients | 1 | 2 | 3 | 4 | 5 | 6 |
| Water | QS 100 | QS 100 | QS 100 | QS 100 | QS 100 | QS 100 |
| Lathering Surfactant: | | | | | | |
| Decyl Polyglucoside | 6 | 6 | 6 | 6 | 6 | 6 |
| Cocamidopropyl | 6 | 6 | 6 | 6 | 6 | 6 |
| Betaine | | | | | | |
| Sodium Lauroyl | 6 | 6 | 6 | 6 | 6 | 6 |
| Sarcosinate | | | | | | |
| Silicone: | | | | | | |
| 10 csk Dimethicone | 20 | — | — | 10 | — | — |
| 1000 csk Dimethicone | — | 20 | — | 10 | — | — |
| 330,000 csk | — | — | 20 | — | — | |
| Dimethicone | | | | | | |
| 50 csk Dimethicone and | — | — | — | — | 20 | — |
| Dimethiconol* | | | | | | |
| Phenyl Trimethicone | — | — | — | — | — | 20 |

*DC-1068 from Dow Corning Corporation

These compositions are then coated at a level of 1 gram onto 6 inch×7.5 inch rectangular pieces of a single-layered hydroentangled/hydroapertured substrate comprising 70% rayon/30% polyester with a basis weight of 70 grams per square meter manufactured by PGI (Chicopee 5763). This substrate has apertures of about 2 mm dispersed within it at a frequency of about 3 apertures per centimeter, and each piece of uncoated substrate weighs approximately 2 grams. The treated substrate is then dried in an oven to constant weight. Alternatively, the treated substrate is allowed to air dry to constant weight.

Alternatively, a single-layered hydroentangled/hydroapertured substrate comprising 70% rayon/30% polyester with a basis weight of 64 grams per square manufactured by BBA, of Simpsonville, S.C. (Nubtex pattern) can be used. This substrate comprises a lattice structure of oriented rows of crisscrossing fibrous bands intersecting at dense fiber nodes, and each uncoated piece of substrate weighs approximately 1.9 grams. The substrate is then dried in an oven to constant weight. Alternatively, the treated substrate is allowed to air dry to constant weight.

The resulting cleansing products are used by wetting with water and are useful for cleansing the skin or hair and for depositing the silicones onto the skin or hair.

### Examples 7-12

The compositions of examples 7-12 are prepared by mixing the following ingredients together in the order shown at room temperature in a suitable vessel:

| | Weight Percent | | | | | |
|---|---|---|---|---|---|---|
| | Example | Example | Example | Example | Example | Example |
| Ingredients | 7 | 8 | 9 | 10 | 11 | 12 |
| Water | QS 100 | QS 100 | QS 100 | QS 100 | QS 100 | QS 100 |
| Lathering Surfactant: | | | | | | |
| Decyl Polyglucoside | 6 | 3 | 6 | 6 | 2 | 6 |
| Cocamidopropyl Betaine | 6 | 3 | 6 | 6 | 8 | 6 |
| Sodium Lauroyl Sarcosinate | 6 | 3 | 6 | 6 | 10 | 6 |
| Silicone: | | | | | | |
| 1000 csk Dimethicone | 2 | 2 | 15 | 15 | 30 | 20 |
| Conditioning Agents: | | | | | | |
| Glycerin | 5 | 5 | 5 | 5 | — | — |
| D-Panthenol | — | — | — | 2 | — | — |
| Niacinamide | — | — | — | 2 | — | — |
| Petrolatum | — | — | 4 | — | — | — |
| Vitamin E Acetate | — | — | — | 1 | — | — |
| Tocopheryl Nicotinate | — | — | 1 | — | — | — |
| Cosmetic Powders: | | | | | | |
| Polyethlene beads* | — | — | — | — | 10 | — |
| PTFE beads** | — | — | — | — | — | 5 |

*Microthene FN-510-00 from Quantam
**Fluoropure 100C from Shamrock

These compositions of examples 7-11 are then coated at a level of 1 gram onto 6 inch×7.5 inch rectangular pieces of single-layered hydroentangled non-apertured 50% rayon/50% polyester substrate with a basis weight of 50 grams per square meter manufactured by PGI (PGI 9950). Alternatively, a single-layered hydroentangled non-apertured 50% rayon/50% polyester substrate with a basis weight of 50 grams per square meter manufactured by BBA (Nubtex pattern), of Simpsonville, S.C., can be used. The treated substrate is then dried in an oven to constant weight. Alternatively, the treated substrate is allowed to air dry to constant weight. After drying, this treated substrate is laminated to a similar sized apertured wet laid paper with 2-3 mm diameter apertures, 3 apertures per centimeter, and with a basis weight of 31 grams per square meter. The total weight of each laminated substrate, without the applied compositions, is approximately 3 grams.

The composition of example 12 is coated in the same fashion as examples 7-11 above, but at a level of 3 grams per piece of substrate. The resulting substrate is then dried and laminated as with examples 7-11 above.

The resulting cleansing products are used by wetting with water and are useful for cleansing the skin or hair and for depositing the silicones and skin conditioning agents onto the skin or hair.

### Examples 13-18

Examples 13-18 are prepared by taking the completed cleansing products of examples 1-6, and then subsequently coating 0.35 grams of petrolatum heated to approximately 70° C. onto each substrate. The petrolatum is coated as an approximately 1 inch wide stripe lengthwise down the center of the substrate, and may be coated onto either the side of the substrate which the cleansing composition was previously applied to, or to the opposite side. The petrolatum on the substrate is then allowed to solidify as it cools under ambient conditions to room temperature, or as the cleansing product is cooled by contacting it with a cold surface or by blowing cold air over it.

The resulting cleansing products are used by wetting with water and are useful for cleansing the skin or hair and for depositing the silicone and conditioning agents onto the skin or hair.

### Examples 19-20

Examples 19 and 20 are prepared by taking the completed cleansing products of example 2, and then subsequently coating 0.35 grams of the following conditioning compositions that have been mixed together and heated to approximately 70° C. onto each substrate.

Weight Percent

| Ingredients | Example 19 | Example 20 |
|---|---|---|
| Conditioning Agent: | | |
| Petrolatum | 90 | 85 |
| Cosmetic Powder: | | |
| Aluminum Starch Octenylsuccinate | 10 | — |
| Silicone Polymer Microbeads* | — | 15 |

*Tospearl 145A from GE

These conditioning compositions are coated as an approximately 1 inch wide stripe lengthwise down the center of the substrate, and may be coated onto either the side of the substrate which the cleansing composition was previously applied to, or to the opposite side. The conditioning composition on the substrate is then allowed to solidify as it cools under ambient conditions to room temperature, or as the cleansing product is cooled by contacting it with a cold surface or by blowing cold air over it.

The resulting cleansing products are used by wetting with water and are useful for cleansing the skin or hair and for depositing the conditioning agents onto the skin or hair.

### Examples 21-25

The compositions of examples 21-25 are prepared by mixing the following ingredients together at room temperature in a suitable vessel:

| | Weight Percent | | | | |
|---|---|---|---|---|---|
| | Example | Example | Example | Example | Example |
| Ingredients | 21 | 22 | 23 | 24 | 25 |
| Water | QS 100 | QS 100 | QS 100 | QS 100 | QS 100 |
| Polyquaternium-10 | 0.5 | 0.25 | — | 0.25 | 0.25 |
| PEG 14M | 1.0 | 0.5 | 0.2 | 0.5 | 0.5 |

Next, the following components are added to the mixture of the above components.

| | | | | | |
|---|---|---|---|---|---|
| Disodium EDTA | 0.10 | 0.10 | 0.10 | 0.10 | 0.10 |
| Decyl Polyglucoside | 6 | 4 | 6 | 6 | 6 |
| Cocamidopropyl Betaine | 6 | 7 | — | — | 6 |
| Sodium | — | — | 6 | — | — |
| Lauroamphoacetate | | | | | |
| Cocamidopropyl Hyroxy- | — | — | — | 6 | — |
| Sultaine | | | | | |
| Sodium Lauroyl Sarcosinate | 6 | 7 | 6 | 6 | 3 |
| Lauroyl Ethylenediamine- | — | — | — | — | 3 |
| triacetic acid, sodium salt | | | | | |
| Methylparaben | 0.2 | 0.2 | 0.2 | 0.2 | 0.2 |
| Phenoxyethanol | 0.2 | 0.2 | 0.2 | 0.2 | 0.2 |
| Benzyl Alcohol | 0.2 | 0.2 | 0.2 | 0.2 | 0.2 |

In a separate mixing vessel, the following components are added. The combination is mixed (with heat to 400 C as necessary) until the propylparaben is dissolved.

| | | | | | |
|---|---|---|---|---|---|
| Water | 2.0 | 2.0 | 2.0 | 2.0 | 2.0 |
| Butylene Glycol | 2.0 | 2.0 | 2.0 | 2.0 | 2.0 |
| Propylparaben | 0.1 | 0.1 | 0.1 | 0.1 | 0.1 |

This mixture is added to the first mixing vessel. Next, the following components are added with mixing to the composition.

| | | | | | |
|---|---|---|---|---|---|
| 1000 csk Dimethicone | 6 | 6 | 6 | 6 | 6 |

These compositions were then coated onto 6 inch×7.5 inch rectangular pieces of single-layered hydroentangled non-apertured 50% rayon/50% polyester substrate with a basis weight of 50 grams per square meter manufactured by PGI (PGI 9950) at a level of 1.5 grams per piece of substrate. The weight of each untreated piece of substrate is approximately 1.5 grams. The treated substrate is then dried in an oven to constant weight. Alternatively, the treated substrate is allowed to air dry to constant weight.

Alternatively, a single-layered hydroentangled non-apertured 70% rayon/30% polyester substrate with a basis weight of 64 grams per square meter manufactured by BBA (Nubtex pattern), of Simpsonville, S.C., can be used. This substrate comprises a lattice structure of oriented rows of criss-crossing fibrous bands intersecting at dense fiber nodes, and each uncoated piece of substrate weighs approximately 1.9 grams. The substrate is then dried in an oven to constant weight. Alternatively, the treated substrate is allowed to air dry to constant weight.

The resulting cleansing products are used by wetting with water and are useful for cleansing the skin or hair and for depositing the conditioning agents onto the skin or hair.

It is understood that the examples and embodiments described herein are for illustrative purposes only and that various modifications or changes in light thereof will be suggested to one skilled in the art without departing from the scope of the present invention.

What is claimed is:

1. A disposable, single use personal care cleansing product comprising:
(a) a water insoluble substrate;
(b) from about 2% to about 400% of a composition, by weight of the substrate, comprising,
(i) a silicone selected from the group consisting of dimethicone, dimethiconol, and mixtures thereof; and
(ii) a lathering surfactant,
wherein (i) and (ii) are in a ratio of from about 1:20 to about 5:1, and wherein the composition is combined prior to being added onto or impregnated into the substrate, and wherein the product is capable of generating an Average Lathering Volume of greater than or equal to about 30 ml.

2. The product according to claim 1, wherein the dimethicone or dimethiconol is nonvolatile.

3. The product according to claim 2, wherein the dimethicone or dimethiconol have viscosities from 10 to 30,000 centistokes.

4. The product according to claim 1, wherein the lathering surfactant is selected from the group consisting of anionic lathering surfactants, nonionic lathering surfactants, amphoteric lathering surfactants, and mixtures thereof.

5. The product according to claim 4, wherein the anionic lathering surfactant is selected from the group consisting of chelating surfactants, sarcosinates, sulfates, isethionates, phosphates, taurates, and mixtures thereof; wherein the nonionic lathering surfactant is selected from the group consisting of amine oxides, alkyl glucosides, alkyl polyglucosides, polyhydroxy fatty acid amides, alkoxylated fatty acid esters, sucrose esters, and mixtures thereof; and wherein the amphoteric lathering surfactant is selected from the group consisting of betaines, sultaines, hydroxysultaines, alkyliminoacetates, iminodialkanoates, aminoalkanoates, and mixtures thereof.

6. The product according to claim 1, wherein the substrate is substantially dry.

7. The product according to claim 1, wherein the substrate comprises one or more materials selected from the group consisting of silks, keratins, celluloses, acetates, acrylics, cellulose esters, modacrylics, polyamides, polyesters, polyolefins, polyvinyl alcohols, and mixtures thereof.

8. The product according to claim 1, wherein the substrate comprises one or more materials selected from the group consisting of wood pulp, cotton, hemp, jute, flax, acrylics, nylons, polyesters, polypropylenes, polyethylenes, polyvinyl acetates, polyurethanes rayon, and mixtures thereof.

9. The product according to claim 1, wherein the substrate is selected from the group consisting of nonwoven substrates, woven substrates, hydroentangled substrates, natural sponges, synthetic sponges, polymeric netted meshes, formed films, thermal bonded substrates, chemical bonded substrates, and mixtures thereof.

10. The product according to claim 9, wherein the substrate comprises two or more layers.

11. The product according to claim 1, further comprising a conditioning component.

12. The product according to claim 11, wherein the conditioning component is selected from the group consisting of esters of fatty acids, polyol polyesters, glycerin mono-esters, glycerin di-esters, glycerin trimesters, epidermal and sebaceous hydrocarbons, lanolin, mineral oil, vegetable oil, vegetable oil adduct, petrolatum, nonionic polymers, glycerin, glycerol, propylene glycol, polypropylene glycols, polyethylene glycols, ethyl hexanediol, hexylene glycols, other aliphatic alcohols, panthenol, urea cationic polymers, polyols, glycolic acid, lactic acid, silicone, vitamin E, vitamin E derivatives, salicylic acid, niacinamide, niacinamide derivatives, and mixtures thereof.

13. The product according to claim 1, further comprising a cosmetic powder.

14. The product according to claim 5, wherein the chelating surfactant is a salt or salts of a hydrophobically modified ethlylenediaminetriacetic acid having the structure 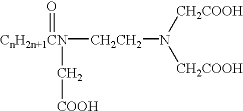
where n is 1 to 40.

15. The product according to claim 1, further comprising an active ingredient.

16. A method of manufacturing the disposable, single use personal cleansing product according to claim 1.

17. A method of cleansing the skin or hair with the product according to claim 1, comprising the steps of:
(a) wetting with water the water insoluble substrate
(b) contacting the skin or hair with the wetted product.
